(19) United States
(12) Patent Application Publication
(10) Pub. No.: US 2024/0252030 A1
(43) Pub. Date: Aug. 1, 2024

(54) LOCKING CARTRIDGE FOR CONNECTING AN ENDOSCOPE TO A VIDEO LARYNGOSCOPE

(71) Applicant: Covidien LP, Mansfield, MA (US)

(72) Inventors: Evan William Tuer, Cupar (GB); Chenghao Bi, Boulder, CO (US); Derek S. Tata, Longmont, CO (US)

(73) Assignee: Covidien LP, Mansfield, MA (US)

(21) Appl. No.: 18/421,525

(22) Filed: Jan. 24, 2024

Related U.S. Application Data
(60) Provisional application No. 63/482,472, filed on Jan. 31, 2023.

(51) Int. Cl. A61B 1/267; A61B 1/00

(52) U.S. Cl. CPC A61B 1/267; A61B 1/00042; A61B 1/00105; A61B 1/00124; A61B 1/00128; A61B 1/00154

(57) ABSTRACT

The technology relates to a cartridge, detachable from a video laryngoscope, that retains a proximal end of an introducer, such as an endoscope. The cartridge includes a housing, an endoscope receptacle at least partially housed within the housing and shaped to receive the proximal end of the endoscope, an electrical interface exposed inside the housing, and an actuator having a locked and unlocked position. Adjustment of the actuator from the unlocked position to the locked position moves the proximal end of the endoscope into contact with the electrical interface and locks the proximal end of the endoscope in the cartridge.

### CROSS-REFERENCE TO RELATED APPLICATIONS

This application claims the benefit of U.S. Provisional Application No. 63/482,472 filed Jan. 31, 2023, entitled “Locking Cartridge for Connecting an Endoscope to a Video Laryngoscope,” which is incorporated herein by reference in its entirety.

### BACKGROUND

Laryngoscopes are commonly used to perform intubations on patients who require breathing assistance. During an intubation, the laryngoscope may be used to manipulate the anatomy of the larynx and associated structures of a patient's airway, in order to obtain a view sufficient for insertion of a breathing tube (e.g., an endotracheal tube) into the trachea. In some situations, the anatomy of the patient, or injury or other health condition of the patient, may prevent a clinician from obtaining a clear view of the larynx. In situations where intubation of a patient may be difficult, an endoscope may be used to aid visualization of the larynx and insertion of the breathing tube. An endoscope is a narrow, flexible tube that typically includes a light and camera at an insertable end of the tube and is inserted into the body for visualizing anatomical structures of a patient. The combined use of a laryngoscope and endoscope may assist the clinician in performing an intubation.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In an aspect, the technology relates to a cartridge, detachable from a video laryngoscope, that retains a proximal end of an endoscope. The cartridge includes a housing; an endoscope receptacle at least partially housed within the housing and shaped to receive the proximal end of the endoscope; an electrical interface exposed inside the housing; and an actuator having a locked and unlocked position. Adjustment of the actuator from the unlocked position to the locked position moves the proximal end of the endoscope into contact with the electrical interface and locks the proximal end of the endoscope in the cartridge.

In an example, the cartridge further includes a drive, and adjustment of the actuator from the unlocked position to the locked position couples a drum of the endoscope with the drive. In a further example, the cartridge also includes a mechanical receiver connected to the drive, wherein the mechanical receiver is exposed on a cartridge surface that contacts the video laryngoscope when the cartridge is attached to the video laryngoscope. In another example, the actuator is a slide. In a further example, the cartridge also includes a mount; and a retention track, wherein as the slide travels along the mount, the retention track causes the endoscope receptacle to move. In another example, the actuator is a knob. In a further example, the cartridge also includes a rotary guide; a retention shaft; and a thread traveler. In yet another example, the actuator is a push button. In a further example, the cartridge also includes a spring coupled to the push button; and a plunger coupled to the spring, wherein depression of the push button causes the plunger to move the proximal end of the endoscope.

In another aspect, the technology relates to a cartridge, detachable from a video laryngoscope, that retains a proximal end of an endoscope. The cartridge includes a receptacle shaped to receive the proximal end of the endoscope; and at least one of a knob, a slide, a push button, or a locking snap, wherein movement of the at least one of the knob, the slide, the push button, or the locking snap from an unlocked position to a locked position causes the proximal end of the endoscope to be locked in the receptacle.

In an example, the cartridge includes an electrical interface, and the movement of the at least one of the knob, the slide, the push button, or the locking snap causes an electrical interface of the endoscope to contact the electrical interface of the cartridge. In a further example, the cartridge includes a drive, and the movement of the at least one of the knob, the slide, the push button, or the locking snap causes a drum of the endoscope to contact the drive. In another example, the cartridge further includes a mechanical stop at an end of the receptacle, wherein when the proximal end of the endoscope contacts the mechanical stop: the drive is aligned with the drum; and the electrical interface of the cartridge is aligned with the electrical interface of the endoscope.

In another aspect, the technology relates to a system for retaining an endoscope in a cartridge, detachable from a video laryngoscope. The system includes an endoscope comprising a steerable distal end and a drive system for steering the steering the steerable distal end; and a cartridge, detachable from a video laryngoscope. The cartridge includes an endoscope receptacle shaped to receive the drive system of the endoscope; a drive; and an actuator having a locked and unlocked position, wherein adjustment of the actuator from the unlocked position to the locked position: moves the drive system of the endoscope into contact with the drive; and locks the drive system of the endoscope in the cartridge. In an example, the actuator is one of a knob, a slide, a push button, or a locking snap.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIGS. 1A-1D depict an example system with a video laryngoscope and an endoscope attached to the video laryngoscope with a detachable locking cartridge.

FIGS. 2A-2C depict another example of a detachable locking cartridge for retaining an endoscope.

FIGS. 3A-3D depict another example of a detachable locking cartridge for retaining an endoscope.

FIG. 4A-4D depict another example of a detachable locking cartridge for retaining an endoscope.

FIG. 5 depicts an example method for retaining an endoscope in a detachable cartridge.

### DETAILED DESCRIPTION

A laryngoscope is commonly used during the intubation of a patient who may require breathing assistance. An intubation is a medical procedure in which a clinician inserts a breathing tube (e.g., an endotracheal tube) into the mouth of the patient, past the larynx, and into the trachea.

The breathing tube may then be connected to a ventilator or other device for supplying breathing gases to the patient. A laryngoscope may be used during intubation to help the clinician manipulate portions of the patient's anatomy, such as the tongue and epiglottis, and obtain a view of the larynx sufficient for inserting the breathing tube into the trachea. To further help visualize the larynx, some laryngoscopes may be configured with a video camera. A laryngoscope that includes a video camera may be referred to as a video laryngoscope (VL), and using a VL to view the larynx or other structures may be referred to as indirect-view laryngoscopy.

With some patients, performing an intubation may be difficult due to a variety of factors, such as inability to position the head or neck of the patient (e.g., due to injury), airway obstruction, atypical anatomy of the patient, other health considerations, or a combination of these or other factors. In these types of scenarios, clinicians may augment the use of a VL with a steerable endoscope, which is a narrow (flexible or rigid) tube that typically includes its own video camera system integrated into a steerable distal tip that is inserted into the patient's body.

The endoscope may be navigated into the airway and positioned such that it provides supplemental visualization of the airway and/or facilitates insertion of the breathing tube. In some examples, the breathing tube is passed over the endoscope into place, with the endoscope itself serving as a channel or guide for inserting the breathing tube. When the breathing tube is placed over the endoscope prior to inserting the endoscope into the patient's airway, the breathing tube is “pre-loaded” onto the endoscope. When the breathing tube is placed over the endoscope after insertion, the breathing tube is “post-loaded”. An endoscope used as a guide for breathing tube insertion (either pre-loaded or post-loaded) may perform the same or similar functions as an introducer and may alternatively be referred to as an introducer in some examples.

The steerable endoscope may include its own display screen as well as an external device for controlling the distal tip, such as a control stick or other type of directional controller. It can be difficult for a single medical professional to view multiple screens and manipulate multiple devices (a video laryngoscope and endoscope) as well as an endotracheal tube during an intubation.

In examples of the present technology, a steerable endoscope may be operatively and removably coupled to a detachable cartridge, which serves as an electrical and mechanical interface between the steerable endoscope and a VL. The detachable cartridge may itself be operatively and removably coupled to the VL. The VL may include one or more electric motors or other actuators for applying steering control, which is mechanically transmitted through the detachable cartridge, to the steerable endoscope. The VL may also include an electrical interface through which electrical power may be provided to the detachable cartridge and/or steerable endoscope, and through which communication signals may be transmitted/received between the VL, detachable cartridge, and steerable endoscope. The ability to disconnect the steerable endoscope, detachable cartridge, and VL from one another allows a clinician to post-load the steerable endoscope, reduces the potential for cross-contamination between the VL and steerable endoscope, facilitates cleaning of the VL, and allows for the detachable cartridge and/or steerable endoscope to be disposable.

The present disclosure further describes techniques for connecting and retaining a steerable endoscope within a cartridge that is detachable from the VL. When the proximal end of the endoscope is inserted into the detachable cartridge and a mechanical control actuator (e.g., knob, slider, snap) is moved to a locked position, elements of the detachable cartridge apply force to the steerable endoscope to retain the steerable endoscope in the detachable cartridge during operation. The applied force may further cause electrical and mechanical elements of the steerable endoscope to engage corresponding electrical and mechanical elements of the detachable cartridge. When the mechanical control actuator is moved to an unlocked position, the endoscope may be removed from the cartridge. Details of these systems and methods are now provided by way of description of the included drawings.

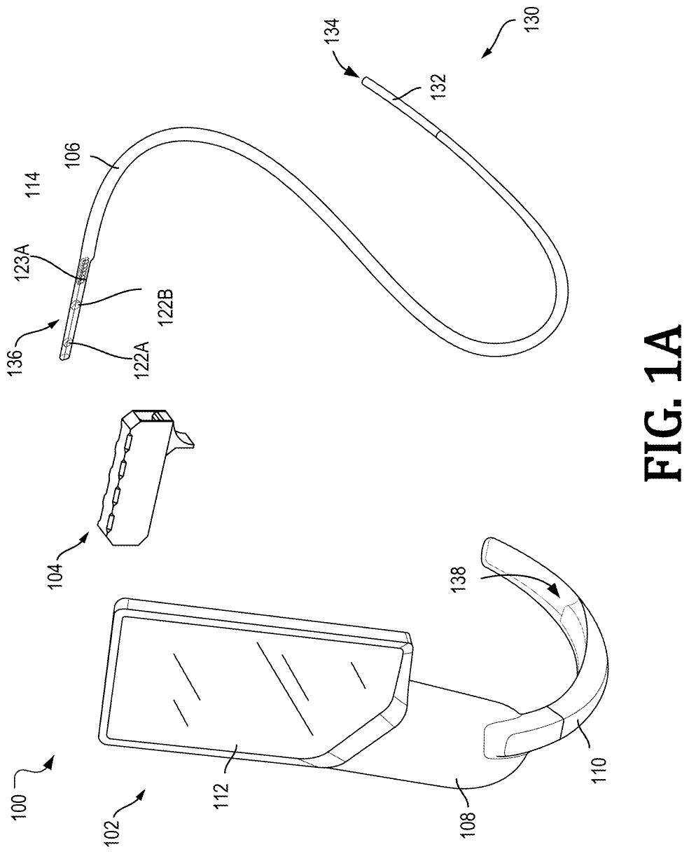

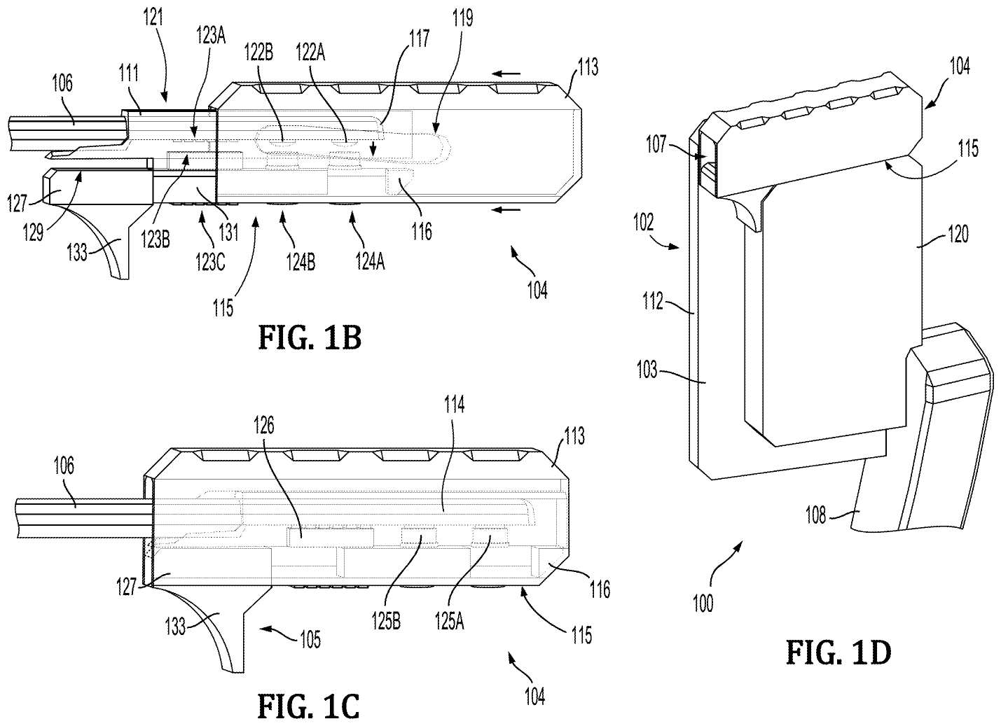

FIG. 1A depicts an example video system 100 with a steerable endoscope 106 having a proximal end 114 and steerable distal end 130. The VL 102 includes a display 112, a handle 108, and arm or extension 110 with a camera 138. On a rear portion of the VL 102 is a detachable cartridge 104, which receives the proximal end 114 of the steerable endoscope 106. At the distal end 130 of the steerable endoscope 106 is a steerable tip 132, which includes an accessory interface 134. In some examples, the accessory interface 134 may include one or more ports for video, lighting, instrument access, and/or other features provided by the steerable endoscope 106. For instance, the accessory interface 134 may be or include a camera. In such examples, the endoscope camera 134 captures endoscope image data.

An endoscope drive system 136 is positioned at the proximal end 114 of the endoscope 106. The endoscope drive system 136 controls or steers a steerable tip 132 of the distal end of the endoscope 106. As one example, the endoscope drive system 136 includes a first drum 122A and a second drum 122B connected to at least two pull wires. In some examples, four pull wires may be included. A first pair of pull wires is configured to move the distal tip in a first plane (e.g., left and right), and a second pair of pull wires is configured to move the tip in a second plane (e.g., up and down) that is substantially orthogonal to the first plane. The first pair of pull wires is connected to opposite sides of the first drum 122A, and the second pair of pull wires is connected to opposite sides of the second drum 122B.

The proximal end 114 of the endoscope 106 also includes an electrical interface 123A, which may include electrical contacts for receiving power and transmitting/receiving signals to/from the detachable cartridge 104 and/or VL 102. The electrical interface 123A provides a source of input power for operating one or more cameras and internal light sources (for viewing the airway) and/or for operating one or more sensors within the endoscope 106, among other functions. The endoscope electrical interface 123A may also provide a data path for transmitting sensor data, video images, and/or other types of data to the detachable cartridge 104 and/or VL 102. For instance, video data captured by the camera of the endoscope 106 may be transmitted to the VL 102 via the endoscope electrical interface 123A.

A detachable cartridge 104 attaches to a rear side of the VL 102 and receives the proximal end 114 of the steerable endoscope 106. The steerable endoscope 106 may be operatively coupled to the detachable cartridge 104 by any of a variety of methods as discussed further herein. When the endoscope proximal end 114 is operatively coupled to the detachable cartridge 104, the electrical interface 123A is conductively connected to a corresponding electrical interface (not depicted in FIG. 1) of the detachable cartridge 104, and the endoscope drive system 136 (e.g., drums 122A-B) engage one or more output drives of the detachable cartridge 104. In addition, when the detachable cartridge 104 is coupled to the VL 102, the VL 102 may transmit steering forces to the detachable cartridge 104 for further transmission to the drums 122A-B of the steerable endoscope 106.

Removing and reconnecting the detachable cartridge 104 and/or steerable endoscope 106, such as to post-load a breathing tube, may increase the risk of exposing the VL 102 to bio-contaminants. For example, when the steerable endoscope 106 is re-inserted into the detachable cartridge 104 following the post-load procedure, any bio-contaminants that may be present on the endoscope proximal end 114 (as a result of the post-load procedure) may be imparted to surfaces of the VL 102. Additionally or alternatively, any dirt or contaminants that may be present on the surface of the VL 102 may be imparted onto the endoscope proximal end 114 as the proximal end 114 is connected/disconnected from the detachable cartridge 104. These contaminants may further be passed to the patient as the breathing tube is post-loaded and guided into the airway. Therefore, the coupling method used to retain the detachable cartridge 104 to the VL 102, along with elements of the electrical and mechanical interfaces (as applicable), may be designed with minimal edges or other physical features that may be prone to trapping dirt or contaminants, and/or the VL 102 may further be designed to be easily and thoroughly cleaned. In some examples, the detachable cartridge 104 and steerable endoscope 106 may be treated as one-time use or disposable, while the VL 102 may be cleaned for re-use. In further examples, the detachable cartridge 104 and steerable endoscope 106 may be sealed and packaged together for one-time use with a VL 102.

FIGS. 1B-D depict an example slide-lock system for connecting a steerable endoscope 106 to a detachable cartridge 104. FIG. 1B depicts an example slide-lock detachable cartridge 104 in an “open” or “unlocked” configuration. FIG. 1C depicts an example slide-lock detachable cartridge 104 in a “closed” or “locked” configuration. FIG. 1D depicts an example VL system 100 with the example slide-lock detachable cartridge 104 connected.

Within the endoscope proximal end 114 is an endoscope electrical interface 123A, which may include electrical contacts for receiving power and transmitting/receiving signals to/from the detachable cartridge 104 and/or VL 102. The electrical interface 123A provides a source of input power for operating one or more cameras and internal light sources (for viewing the airway), and for operating one or more sensors within the steerable endoscope 106, among other functions. The endoscope electrical interface 123B electrically couples with a cartridge-endoscope electrical interface 123B in the detachable cartridge 104 when the endoscope 106 is connected to the cartridge 104.

The electrical interfaces 123A-B may include a plurality of conductive pads, receptacles, pins, balls, ports, and/or other conductive elements that may be caused to contact and electrically connect to corresponding and opposing elements of the cartridge-endoscope electrical interface 123B. In some examples, the endoscope electrical interface 123A may include male—or female-type elements, and cartridge-endoscope electrical interface 123B may include female—or male-type elements, respectively. In other examples, one or more of the elements of one of the electrical interfaces 123A or 123B may include flexible or spring-loaded pins (such as pogo pins) and corresponding elements of the opposing electrical interface 123B or 123A, respectively, may include conductive pads or other elements for receiving and contacting these spring-loaded pins. In still other examples, a wide array of electrical interface technologies may be utilized to establish electrical connectivity between the endoscope electrical interface 123A and cartridge-endoscope electrical interface 123B.

To open the detachable cartridge and connect the steerable endoscope, a cartridge slide 113 is slid away from an external portion 131 of a cartridge mount 127 to open the cartridge 104. For instance, the cartridge mount 127 may include an internal portion 131 (that is internal to the cartridge slide 113 when the cartridge 104 is closed) and an external portion 133 (that is external to the cartridge slide 113 when the cartridge 104 is open). The cartridge slide 113 has a cavity and opening to the cavity that receives the internal portion 131 of the cartridge mount 127. For instance, the internal portion 131 may be an elongate rod or body that extends from the external portion 133 of the cartridge mount 127.

The cartridge slide 113 is slid until hitting a mechanical stop of the cartridge slide 113 that abuts another mechanical stop of the detachable cartridge 104. The cartridge slide 113 and/or other elements of the detachable cartridge 104 may include features such as rails, lips, guides, tracks, and/or other features that restrict the movement of the cartridge slide 113 to straight-line (sliding) travel between mechanical stops of the detachable cartridge 104.

When the cartridge slide 113 is extended away from the external portion 133 of the cartridge mount 127, the cartridge slide 113 and detachable cartridge 104 are considered to be “open” or “unlocked,” as depicted in FIG. 1B. With the cartridge slide 113 in the unlocked position, an endoscope receiver or receptacle 111 is exposed, along with an unlock indicator 121, which provides visual feedback that the detachable cartridge 104 is unlocked and may receive the endoscope proximal end 114. In some examples, the unlock indicator 121 may be included on an outer surface of the endoscope receiver 111, such as on a top surface (as indicated in FIG. 1B), and/or the unlock indicator 121 may be included one or more other surfaces of the detachable cartridge 104. In examples, the unlock indicator 121 may be printed, etched, stamped, or otherwise affixed to one or more surfaces of the detachable cartridge 104, and may be depicted as an open lock symbol, printed text, or other symbol or rendering that indicates the detachable cartridge 104 is unlocked and may receive the endoscope proximal end 114.

With the detachable cartridge 104 in the unlocked configuration, the endoscope proximal end 114 may be fully inserted into the endoscope receiver 111 through an endoscope port 107, where a portion of the endoscope proximal end 114 abuts the receiver stop 117. The endoscope receiver 111 may be an elongate rigid body defining an elongate cavity that is sized and shaped to receive the proximal end of the endoscope 106. In examples, the receiver stop 117 may be a mechanical stop that prevents further insertion of the endoscope proximal end 114 and aligns the endoscope drive system 136 (e.g., drums 122A-B) with cartridge drives 125A-B, respectively, and aligns the endoscope electrical interface 123A with a cartridge-endoscope electrical interface 123B. In one example, the receiver stop 117 may be the terminus of the cavity of the endoscope receiver 111 that accepts the endoscope proximal end 114. In another example the receiver stop 117 may be a defined mechanical stop or other element. The position of the receiver stop 117 may be configured such that the drums 122A-B and electrical interfaces 123A-B are properly aligned when the proximal end of the endoscope 106 is fully inserted into the cartridge 104.

The endoscope proximal end 114 may include additional features that interact with and engage counter-facing features that may be included in the endoscope receiver 111 to further cause or ensure alignment between electrical/mechanical elements of the endoscope proximal end 114 with corresponding electrical/mechanical elements of the detachable cartridge 104. In one example, the endoscope proximal end 114 may include one or more rails and the endoscope receiver 111 may include one or more guides for receiving the rails. The guides may permit insertion of the endoscope proximal end 114 only when the rails are aligned with the guides, such that corresponding and opposing electrical/mechanical elements of the detachable cartridge 104 and steerable endoscope 106 are caused to face each other. In another example, the shape of the endoscope proximal end 114, and/or the shape of elements or features of the endoscope receiver 111 may also be designed to allow insertion of the endoscope proximal end 114 in only one orientation. In other examples, one or more features, such as slots, tracks, notches, keyed elements, and/or other features of the endoscope proximal end 114 and/or endoscope receiver 111 may restrict the insertion direction of the endoscope proximal end 114 to prevent incorrect insertion.

With the endoscope proximal end 114 fully inserted (in the correct orientation) into the endoscope receiver 111, the cartridge slide 113 is slid toward the external portion 133 of the cartridge mount 127, as indicated by the left-facing arrows depicted in FIG. 1B. The cartridge slide 113 may include a retention track 119 that engages the endoscope receiver 111. In examples, the retention track 119 may be a recess (or protrusion) that is sloped, angled, curved, or otherwise configured to apply force to the endoscope receiver 111 as the cartridge slide 113 is caused to move toward the external portion 133 of the cartridge mount 127. For instance, the endoscope receiver 111 may include a protrusion (or recess) that slides in the retention track 119 as the cartridge slide 113 is moved. The applied force causes the endoscope receiver 111 (and fully inserted endoscope proximal end 114) to advance toward a mating surface 129 of the cartridge 127, as indicated by the downward-facing arrow in FIG. 1B. The detachable cartridge 104 may be designed so that an interior portion of the cartridge slide 113 abuts a slide stop 116 when at least a portion endoscope receiver 111 contacts the mating surface 129. When an interior portion of the cartridge slide 113 abuts the slide stop 116, as depicted in FIG. 1C, the cartridge slide 113 and detachable cartridge 104 are considered to be “closed” or “locked.”

The endoscope receiver 111, cartridge slide 113, and/or other elements of the detachable cartridge 104 may include a retention feature, such as a detent, that applies static force to maintain the cartridge slide 113 in the locked position. The cartridge slide 113 may be maintained in the locked position until sufficient force is applied to the cartridge slide 113 to overcome the static force of the detent and enable travel of the cartridge slide 113 towards the unlocked position.

While locked, elements of the endoscope electrical interface 123A make electrical contact with corresponding elements of the cartridge-endoscope electrical interface 123B. The first drive 125A and second drive 125B are mechanically engaged with endoscope first drum 122A and second drum 122B, respectively. The drives 125A-B and/or drums 122A-B may include force-transmitting/force-receiving features, such as edges, surfaces, corners, prongs, notches, studs, teeth, cogs, protrusions, and/or other features that facilitate the transmission of steering forces from the drives 125A-B to the drums 122A-B.

The cartridge drives 125A-B and/or drums 122A-B may further include alignment features that automatically align the drums 122A-B and cartridge drives 125A-B during closure of cartridge slide 113. For example, the cartridge drives 125A-B and/or drums 122A-B may include guides, slots, chamfers, bevels, and/or other types of alignment features that automatically cause the force-transmitting features of the drives 125A-B to align and engage force-receiving features of the drums 122A-B. In some examples, the force-transmitting/force-receiving features may themselves include the alignment features, while in other examples the force-transmitting/force-receiving features may be implemented separately from the alignment features.

With the cartridge slide 113 in the locked position, elements of the detachable cartridge may exert force on a portion of the endoscope proximal end 114 to cause the endoscope proximal end 114 to be retained within the endoscope receiver 111. For example, the retention track 119 may cause static force to be applied to the endoscope receiver 111, which in turn may apply a clamping, squeezing, or other type of retention force to the endoscope proximal end 114. Additionally or alternatively, engagement of the endoscope drums 122A-B and the cartridge drives 125A-B may cause retention of the endoscope proximal end 114 in the detachable cartridge 104, since engagement of the drums 122A-B and the cartridge drives 125A-B may physically/mechanically prevent the endoscope proximal end 114 from inadvertently being fully or partially withdrawn from the endoscope receiver 111.

To remove the steerable endoscope 106 from the detachable cartridge 104, the cartridge slide 113 slid away from the external portion 133 of the cartridge mount 127. The retention track 119 engages the endoscope receiver 111, causing the endoscope receiver 111 to be drawn away from the mating surface 129 as the cartridge slide 113 is extended. When the cartridge slide 113 is fully extended, a mechanical stop of the cartridge slide 113 abuts another mechanical stop of the detachable cartridge 104 and the detachable cartridge 104 is considered to be unlocked. Extension of the cartridge slide 113 releases retention force applied to the endoscope proximal end 114, allowing the steerable endoscope 106 to be removed from the detachable cartridge 104.

In some examples, the detachable cartridge 104 may include one or more spring elements that facilitate the locking and/or unlocking of the detachable cartridge 104. For example, the endoscope receiver 111 may be associated with one or more spring elements that compress when the cartridge slide 113 is caused to close/lock, and release when the cartridge slide 113 is caused to open/unlock. The spring element(s) may apply force to the endoscope receiver 111 to facilitate travel of the endoscope receiver 111 away from the mating surface 129, as the cartridge slide 113 is extended during opening/unlocking. In other examples, the detachable cartridge 104 may include spring elements and/or other mechanical elements that engage one or more elements of the detachable cartridge 104 to facilitate locking and/or unlocking.

Within the detachable cartridge 104, the cartridge drives 125A-B are each mechanically coupled to a first and second mechanical receiver 124A-B, respectively. For example, the cartridge drives 125A-B may be coupled to the mechanical receivers 124A-B by one or more axles, gears, spindles, linkages, and/or other mechanical elements. The mechanical receivers 124A-B may include similar force-receiving features as described above for engaging steering control elements of the VL 102. The mechanical receivers 124A-B may further include alignment features such as guides, slots, chamfers, bevels, and/or other types of alignment features that automatically cause the force-transmitting features of the VL 102 to align and engage force-receiving features of the mechanical receivers 124A-B.

The detachable cartridge 104 also includes a cartridge-VL electrical interface 123C, which includes one or more of the electrical elements described above that electrically connect to corresponding and opposing electrical elements of the VL 102. Electrical connection may be established between the cartridge-endoscope electrical interface 123B and cartridge-VL electrical interface 123C via electrical passthrough 126. The electrical passthrough may include any of a variety of elements suitable for electrical connection, such as wires, rigid or flexible pins, flexible (flex) printed circuits, rigid or rigid-flex printed circuit boards (PCBs), and/or other elements suitable for establishing electrical connection.

The cartridge-VL electrical interface 123C and the mechanical receivers 124A-B may protrude from, be recessed in, or be exposed on a cartridge interface surface 115 of the cartridge mount 127. The cartridge interface surface 115 may be part of the internal portion 131 of the cartridge mount 127. For example, the electrical passthrough 126 may pass through the internal portion 131 of the cartridge mount 127. Similarly, the cartridge drives 125A-B extend through the internal portion 131 of the cartridge mount 127.

FIG. 1D depicts an example VL system 100, with the detachable cartridge 104 (in the locked configuration) coupled to the VL 102. When coupled, at least a portion of the detachable cartridge 104 may abut or contact a rear surface 103 of the VL 102. In addition, portions of a cartridge interface surface 115 and a cartridge-mount inner surface 105 may abut surfaces of a drive housing 120 that protrudes from a rear surface of the VL 102. For example, the VL 102 may include a display housing that houses the display 112 at the front surface, and the drive housing 120 protrudes from the rear surface of the display housing. The drive housing 120 may be in the form of, or include, a compartment, gear box, drive chain, transformer, or other similar items. While the drive housing 120 is depicted as being substantially rectangular, in other examples the drive housing may be shaped differently. The cartridge mount inner surface 105 is a surface of the exterior portion 133 of the cartridge mount 127. The cartridge interface surface 115 and the cartridge mount inner surface 105 may be substantially orthogonal to one another. The cartridge interface surface 115 may contact a first surface of the drive housing 120, and the cartridge mount inner surface 105 may contact another surface of the drive housing 120.

In some examples, the cartridge interface surface 115, cartridge mount inner surface 105, drive housing 120, other portions of VL 102, and/or other portions of detachable cartridge 104 may include one or more magnetic elements (such as static, permanent magnets) that apply retention force(s) between the VL 102 and detachable cartridge 104, in order to keep the detachable cartridge 104 affixed to the VL 102. The magnet(s) may also apply force that causes elements of the cartridge-VL electrical interface 123C and mechanical drives 124A-B to align with corresponding and opposing elements of the VL 102 when the detachable cartridge is brought within proximity of the VL 102 for connection.

In other examples, the detachable cartridge 104 may be aligned and retained to the VL 102 by other methods, such as by spring retention elements and/or other types of retention elements. The VL 102 and/or detachable cartridge 104 may include alignment and/or retention features, such as guides, rails, notches, posts, or other physical features that facilitate alignment and retention of the detachable cartridge 104 to the VL 102. In some examples, when physical features, external to the VL 102 and detachable cartridge 104, are used to align and/or retain the detachable cartridge 104 to the VL 102, the features may be designed with minimal recesses that trap dirt, bio-contaminants, pathogens, and/or other contaminants. Such an approach may facilitate cleaning of the VL 102 for re-use.

The drive housing 120 may include mechanical elements for providing steering control of the endoscope steerable tip. For instance, the drive housing 120, or other portion of the VL 102, may include one or more motors, actuators, axles, gears, spindles, linkages, and/or other elements for generating and transmitting steering forces to the endoscope steerable tip, via the detachable cartridge 104. In some examples, the VL 102 may include one motor (and associated mechanical elements) for each of the drums 122A-B of the steerable endoscope 106, such that each motor may control movement of the endoscope steerable tip along one or more planes.

The drive housing 120 may further include electrical elements associated with the provision of electrical power, such as one or more batteries, voltage regulators, current sources, protection elements, and/or other elements associated with the provision of electrical power. These elements may provide electrical power to the detachable cartridge 104 and/or steerable endoscope 106, in addition to providing electrical power to elements of the VL 102. The VL 102 may include one or more processors, memory elements, display controllers. user interface elements, sensors, analog circuits, digital circuits, and/or other electrical elements and components associated with the function and operation of the VL 102, detachable cartridge 104, and steerable endoscope 106.

In some examples, the drive housing 120 may not be protruding from the rear of the VL 102 (e.g., the drive housing 120 may be omitted). Rather, the mechanical elements and electrical elements of the drive housing 120 discussed above may be integrated into the rear surface of the VL 102.

The VL 102 may receive data from the steerable endoscope 106 via the electrical interfaces 123A-C. For example, the VL 102 may receive sensor data (such as from a positional sensor of the steerable endoscope 106), video data (such as from a video camera system associated with the steerable endoscope 106), and/or other data transmitted to the VL 102. For instance, data generated by the endoscope 106 may be transferred to the VL 102 by passing the data from the endoscope electrical interface 123A to the cartridge-endoscope electrical interface 123B, which then gets passed through the cartridge-VL electrical interface 123C to a laryngoscope electrical interface of the VL 102. Data and/or control signals may also be passed from the VL 102 to the cartridge 104 and/or the endoscope 106 via the same electrical interfaces.

Video images received by the VL 102 from a video camera of the steerable endoscope 106 (through electrical interfaces 123A-C) may be provided on display 112, as the distal end of the steerable endoscope 106 is navigated through a patient's airway. Steering control inputs may be received by the VL 102 (such as through display 112 or other directional controller) and translated to signals that control the steering forces generated by elements of the VL (such as by motors or other actuators). Elements of the VL 102 may transmit the steering forces to the cartridge mechanical receivers 124A-B, which further transmit the steering forces to the cartridge drives 125A-B, and ultimately to the endoscope drums 122A-B. Thus, the orientation of the endoscope steerable tip 132 may be controlled during navigation through the patient airway, and/or during or following intubation.

In examples, the VL 102, detachable cartridge 104, and steerable endoscope 106 may be connected and disconnected in any order, at any time during operation. For example, the steerable endoscope 106 may be removed from the detachable cartridge 104 in order to post-load a breathing tube, while the detachable cartridge 104 remains coupled to the VL 102. The steerable endoscope 106 may be reinserted into the detachable cartridge 104 following the post-load procedure. In another example, the detachable cartridge 104 may be decoupled from the VL 102 while the steerable endoscope 106 remains coupled to the detachable cartridge 104.

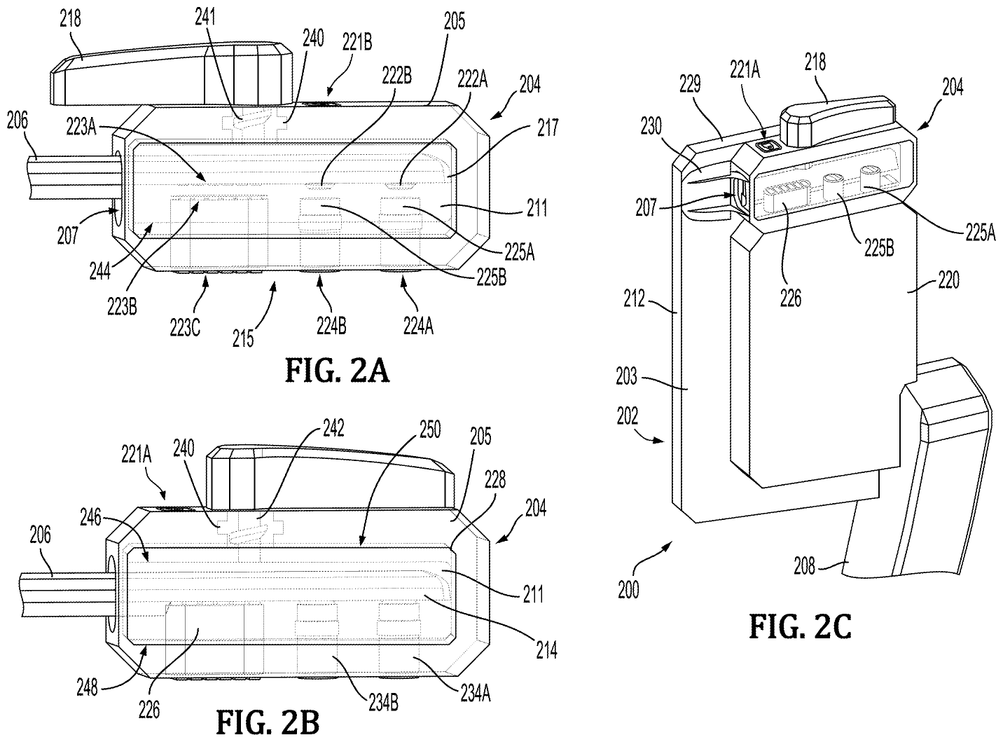

FIGS. 2A-C depict another example of a detachable cartridge 204 that uses a twist-lock system for connecting a steerable endoscope 206 to the detachable cartridge 204. FIG. 2A depicts an example twist-lock detachable cartridge 204 in an open or unlocked configuration. FIG. 2B depicts an example twist-lock detachable cartridge 204 in a closed or locked configuration. FIG. 2C depicts an example VL system 200 with the example twist-lock detachable cartridge 204 connected with a back cover of the cartridge 204 removed.

Elements of the detachable cartridge 204 and steerable endoscope 206 may be similar to, or the same as, corresponding elements depicted in FIGS. 1A-C. For example, elements of the electrical interfaces 223A-C and electrical passthrough 226 may be similar to, and/or function the same as, electrical interfaces 123A-B and electrical passthrough 126. The electrical interfaces 223A-C and electrical passthrough 226 distribute power from the VL 202 to electrical elements of the detachable cartridge 204 and steerable endoscope 206 and provide an electrical signal path for communication between the VL 202, detachable cartridge 204, and steerable endoscope 206 (such as for the transmission/reception of video images, sensor data, etc.). Further, steering control elements, such as drums 222A-B, receivers 224A-B, and drives 225A-B may be similar to, and/or function the same as the corresponding elements of the detachable cartridge 104 discussed above, and they may protrude from or be exposed on a cartridge interface surface 215. FIGS. 2A-B depict mechanical couplings 234A-B which connect cartridge drives 225A-B to mechanical receivers 224A-B, respectively. In this example, the mechanical couplings 234A-B are rigid cylindrical elements that transmit steering forces applied to the mechanical receivers 224A-B directly to the cartridge drives 225A-B. In other examples, the mechanical couplings 234A-B may each include one or more axles, gears, and/or other elements suitable for directly or indirectly transmitting steering forces from the mechanical receivers 224A-B to the cartridge drives 225A-B.

The detachable cartridge 204 has an external housing 205 that houses an endoscope receiver or receptacle 211, which accepts the endoscope proximal end 214 that is inserted into the endoscope receiver 211 through an endoscope port 207. The endoscope receiver 211 includes a receiver stop 217 that is abutted by the endoscope proximal end 214 when the endoscope proximal end 214 is fully inserted into the endoscope receiver 211. As described for FIGS. 1A-B, the endoscope proximal end 214 and endoscope receiver 211 may include features (such as rails, guides, keyed elements, etc.) that permit insertion of the endoscope proximal end 214 in only one orientation, such that electrical interfaces 223A-B, and drums 222A-B and cartridge drives 225A-B are facing each other, respectively, and aligned for engagement.

FIG. 2A depicts the detachable endoscope 204 in the unlocked position, in which an unlock indicator 221B may be visible. With the endoscope proximal end 214 fully inserted into the endoscope receiver 211, the detachable cartridge may be adjusted the closed or locked configuration, as depicted in FIG. 2B, by rotating a retention knob 218. Rotating the retention knob 218 to the closed position may cause the unlock indicator 221B to be covered by the retention knob 218. In some examples, rotation of the retention knob 218 may also reveal a locked indicator 221A, which may be similar to the unlock indicator 221B.

The retention knob 218 may be connected to a rotary guide 240 that rotates with the retention knob 218. The rotary guide 240 may include one or more threads (not depicted for visual clarity), located on the interior of the rotary guide 240, that engage a thread traveler 241, which is connected to a retention shaft 242. The thread traveler 241 may be angled to match the pitch of the interior threads of the rotary guide 240. Rotation of the retention knob 218 causes rotation of the rotary guide 240, which exerts force on the thread traveler 241. The applied force causes linear movement of the thread traveler 241 in a direction parallel to the retention shaft 242 and towards the chamber lower surface 248, causing the retention shaft 242 to extend in a direction towards the chamber lower surface 248.

The retention shaft 242 is further connected to, or in contact with, a portion of the endoscope receiver 211, which is enclosed within an inner chamber 228 of the detachable cartridge 204. For instance, a portion of the endoscope receiver 211 is positioned within an inner chamber 228 defined by the external housing 205 of the cartridge 204. In some examples, the retention shaft 242 may be directly connected to the endoscope receiver 211, while in other examples the retention shaft 242 may be indirectly connected to the endoscope receiver 211 via gears, axles, and/or other mechanical elements.

The dimensions of the inner chamber 228 may be chosen to allow limited, bidirectional travel of the endoscope receiver 211 in directions parallel to the retention shaft 242. In examples, the endoscope receiver 211 and/or inner chamber 228 may include features, such as guides, rails, grooves, and/or other features associated with restricting, directing, and/or facilitating bidirectional travel of the endoscope receiver 211.

Extension of the retention shaft 242 causes the endoscope receiver 211 (and fully inserted endoscope proximal end 214) to advance towards the chamber lower surface 248 to cause electrical interfaces 223A-B to be in electrical contact and cause endoscope drums 222A-B to mechanically engage cartridge drives 225A-B, respectively. The endoscope receiver 211 and/or inner chamber 228 may include additional features that prevent further travel of the endoscope receiver 211 once sufficient electrical contact and mechanical engagement is established. In some examples, the endoscope receiver 211 and/or inner chamber 228 may include one or more dedicated or defined mechanical stops that prevent further travel of the endoscope receiver 211 towards the chamber lower surface 248. In other examples, the endoscope receiver 211 and/or inner chamber 228 may be designed such that at least a portion of the receiver lower surface 244 abuts at least a portion of the chamber lower surface 248 (as depicted in FIG. 2B) once sufficient electrical contact and mechanical engagement is established.

When the electrical interfaces 223A-B are electrically connected, and drums 222A-B engaged by cartridge drives 225A-B, the detachable cartridge 204 is considered to be closed or locked. When the detachable endoscope 204 is locked, elements of the detachable cartridge 204 may apply retention force to at least a portion of the endoscope proximal end 214, to prevent unintended detachment of the endoscope proximal end 214 from the detachable cartridge 204. The endoscope receiver 211 and/or other element of the detachable cartridge 204 may apply retention force (such as clamping, squeezing, etc.), to retain the endoscope proximal end 214. Additionally or alternatively, the engagement of the drums 222A-B and cartridge drives 225A-B may physically/mechanically prevent the endoscope proximal end 214 from inadvertently being fully or partially withdrawn from the detachable cartridge 204.

The detachable cartridge 204 may include features that further indicate that the detachable cartridge 204 is fully closed or locked. For example, elements of the detachable cartridge 204 (such as the rotary guide 240, thread traveler 241, etc.) may include a detent or similar feature that provides haptic feedback that the retention knob 218 is positioned in the lock configuration. The detent may also provide a static force that prevents inadvertent rotation of the retention knob 218 out of the locked position (such as described for FIG. 1C). In some examples, rotating the retention knob 218 or other elements of the detachable cartridge 204 may provide a haptic response such as a click, and/or may provide an audible indicator that the retention knob 218 is in the locked configuration. In other examples, one or more mechanical stops of the detachable cartridge 204 (such as when the endoscope receiver 211 abuts the chamber lower surface 248) may prevent further rotation of the retention knob 218 and may be used to indicate that the detachable cartridge 204 is locked.

In further examples, the position of the retention knob 218 itself may further indicate that the detachable cartridge 204 is locked. For instance, the detachable cartridge 204 may be designed to achieve lock with a 90-degree rotation of the retention knob 218 (relative to the locked position). In other examples, rotation of the retention knob 218 by 180 degrees, as depicted in FIGS. 2A-B, may indicate that the detachable cartridge 204 is locked. When the retention knob 218 is rotated by 90 degrees, 180 degrees, or by another amount of rotation, the detachable cartridge 204 may provide one or more of the indications described above, that the detachable cartridge 204 is locked.

To remove the steerable endoscope 206 from the detachable cartridge 204, the retention knob 218 may be rotated to the unlocked position depicted in FIG. 2A. Rotation of the retention knob 218 toward the unlocked position causes rotation of the rotary guide 240 such that the thread traveler 241 and retention shaft 242 are caused to be retracted in the direction of the retention knob 218. Retraction of the retention shaft 242 causes the endoscope receiver 211 to be retracted from the chamber lower surface 248, towards the chamber upper surface 250. Retraction of the endoscope receiver 211 releases retention force that may have been applied to the endoscope proximal end 214 to be released, allowing the steerable endoscope 206 to be removed from the detachable cartridge 204.

In some examples, the detachable cartridge 204 may include one or more spring elements that facilitate the locking and/or unlocking of the detachable cartridge 204. For example, the endoscope receiver 211 may be associated with one or more spring elements that are compressed when the retention knob 218 is rotated into the locked position and released when the retention knob is rotated to the unlocked position. The spring element(s) may apply force to the endoscope receiver 211 to facilitate travel of the endoscope receiver 211 away from the chamber lower surface 248, as the retention knob 218 is rotated during opening/unlocking. In other examples, the detachable cartridge 204 may include spring elements and/or other mechanical elements that engage one or more elements of the detachable cartridge 204 to facilitate locking and/or unlocking.

As described above for the scenario where the detachable cartridge 204 is transitioned to the locked configuration, elements of the detachable cartridge 204 may include one or more mechanical stops that prevent further travel of the endoscope receiver 211 towards the retention knob 218 when the retention knob 218 reaches the unlocked position. For example, the receiver upper surface 246 may abut the chamber upper surface 250, when the detachable cartridge 204 is in the unlocked position. When unlocked, the endoscope proximal end 214 may be removed from the detachable cartridge 204.

FIG. 2C depicts the detachable cartridge 204 (with steerable endoscope 206 removed) in the locked configuration connected to the VL 202. The VL 202 may include similar features and functions as the VL 102 described above. For example, the VL 202 may include a display 212 (which may be a touch-sensitive display), drive housing 220, handle 208, and other elements that perform similar, or the same, functions as the corresponding elements described for FIG. 1C. The VL 202 includes an electrical interface that electrically connects to the cartridge-VL electrical interface 223C when the detachable cartridge is connected. The VL 202 may further include mechanical drive elements that engage cartridge mechanical receivers 224A-B and transmit steering forces to the steerable endoscope 206 via the detachable cartridge 204.

The detachable cartridge 204 and/or VL 202 may be designed such that when the detachable cartridge is connected to the VL 202, the retention knob 218 may be rotated above the VL top surface 229. In examples where the detachable cartridge 204 and/or VL 202 are designed to provide sufficient clearance of the retention knob 218 above the top surface 229, the detachable cartridge 204 may allow rotation of the retention knob 218 in the clockwise, counterclockwise, or both directions. That is, the detachable cartridge 204 may be designed for left-handed, right-handed, or both types of rotation of the retention knob 218.

The VL 202 and/or detachable cartridge 204 may include features and/or elements that facilitate alignment and retention of the detachable cartridge 204 to the VL 202. As described above regarding the cartridge 104, the VL 202 and/or detachable cartridge 204 may include permanent magnets and/or other features that apply alignment and retention force(s) between the VL 202 and detachable cartridge 204, in order to keep the detachable cartridge 204 affixed to the VL 202.

The rear surface 203 of the VL 202 may include a contamination guard 230, which may reduce the potential for bio-contaminants to be transferred between the VL 202, detachable cartridge 204, and steerable endoscope 206. The contamination guard 230 may be detachable from the rear surface 203 and disposed of following use, such as after completion of an intubation. In some examples, the contamination guard 230 may be detached from the rear surface 203 and cleaned, wiped, sanitized, sterilized, etc., for reuse. In other examples, the contamination guard 230 may remain affixed to the rear surface 203 following use and appropriately cleaned. In still other examples, the contamination guard may be permanently, semi-permanently, or detachably affixed to the detachable cartridge 204. In one example, the contamination guard 230 may be affixed to, and packaged with the detachable cartridge 204 for one-time-use. For instance, the contamination guard 230 may include a planar surface that protrudes from the external housing 205 of the detachable cartridge 204.

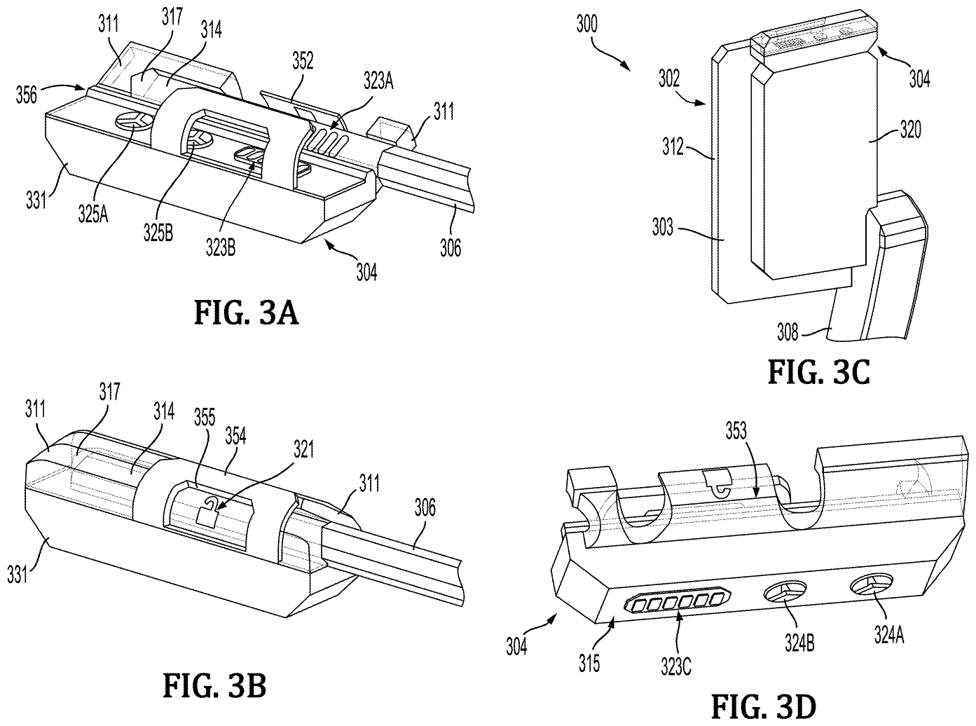

FIGS. 3A-D depict another example of a detachable cartridge 304 that uses a snap-lock system for connecting a steerable endoscope 306 to a detachable cartridge 304. FIG. 3A depicts an example snap-lock detachable cartridge 304 in an open or unlocked configuration. FIG. 3B depicts an example snap-lock detachable cartridge 304 in a closed or locked configuration. FIG. 3C depicts an example VL system 300 with the example twist-lock detachable cartridge 304 connected. FIG. 3D depicts a perspective view of the bottom surface of the example snap-lock detachable cartridge 304.

Elements of the detachable cartridge 304 and steerable endoscope 306 may be similar to, or the same as, corresponding elements depicted in FIGS. 1A-D and/or FIGS. 2A-C. For example, cartridge 304 includes electrical interfaces 323A-C that may be similar to, and/or function the same as, electrical interfaces 123A-C and/or 223A-C. Though not explicitly depicted in FIGS. 3A-D, the detachable cartridge 304 may also include an electrical passthrough, that may be the same as, or similar to, electrical passthrough 226. The electrical passthrough electrically connects electrical interfaces 323B-C within the detachable cartridge 304. The electrical interfaces 323A-B and electrical passthrough distribute power from the VL 302 to electrical elements of the detachable cartridge 304 and steerable endoscope 306 and provide an electrical signal path for communication between the VL 302, detachable cartridge 304, and steerable endoscope 306 (such as for the transmission/reception of video images, sensor data, etc.).

Further, steering control elements, such as mechanical receivers 324A-B and cartridge drives 325A-B, of the detachable cartridge 304 may be similar to and/or function similar to the steering control elements 124A-B/224A-B and 125A-B/225A-B. While not depicted, the steerable endoscope 306 also includes a drive system such as drums that may be similar to, or the same as, drums 122A-B and/or 222A-B, which may be mechanically linked to, and provide for control of, the endoscope steerable tip.

At least one difference between the detachable cartridge 304, and the detachable cartridges 104 and 204 described above, is the snap-lock feature by which the endoscope proximal end 314 is inserted into, and retained within, the detachable cartridge 304. As depicted in FIG. 3A, an endoscope receiver or receptacle 311 is connected to a cartridge base 331 by a receiver hinge 356. In the open or unlocked configuration, the endoscope proximal end 314 is positioned within the endoscope receiver 311 with the endoscope electrical interface 323A and drums facing away from endoscope receiver 311. The endoscope proximal end 314 is positioned such that the endoscope proximal end 314 abuts a receiver stop 317 of the endoscope receiver 311.

In examples, the endoscope receiver 311 and/or endoscope proximal end 314 may each be shaped so that the endoscope proximal end 314 may be positioned within the endoscope receiver 311 in only one orientation, with the endoscope electrical interface 323A and drums facing away from the outer surface of endoscope receiver 311. In some examples, the endoscope receiver 311 and/or endoscope proximal end 314 may further include alignment features, such as posts, keyed elements, and/or other features that ensure correct orientation and/or position of the endoscope proximal end 314 within the endoscope receiver 311.

To ensure that the endoscope proximal end 314 is secured within the endoscope receiver 311, the endoscope receiver 311 and/or endoscope proximal end 314 may be designed such that retention force is applied to the endoscope proximal end 314 when positioned within the endoscope receiver 311. For example, the endoscope proximal end 314 may be press-fit, snap-fit, or seated into the endoscope receiver 311, where compressive forces exerted between the endoscope receiver 311 and endoscope proximal end 314 may cause the endoscope proximal end 314 to be retained within the endoscope receiver 311.

With the endoscope proximal end 314 positioned within the endoscope receiver 311, the endoscope receiver 311 may be rotated about the receiver hinge 356 into the closed or locked position depicted in FIG. 3B. During rotation, a locking snap 352 of the endoscope receiver 311 may interfere and encounter friction with the snap receiver 354 of the cartridge base 331. For instance, the locking snap 352 may be a protrusion, and the locking receiver 354 may be a recess or hole. In other examples, the locking snap 352 may be a recess and the locking receiver 354 may be a protrusion. The force applied to the endoscope receiver 311 to cause rotation may further cause deflection of the locking snap 352 and/or snap receiver 354, during rotation, due to the interference. Upon further rotation of the endoscope receiver 311, portions of the endoscope receiver 311 may abut the cartridge base 331 as the snap detent 353 rotates under and past the snap engagement lip 355 of the snap receiver 354. Once the snap detent 353 has cleared the snap engagement lip 355, the interference between the locking snap 352 and snap receiver 354 is released, allowing the deflection of the locking snap 352 and/or snap receiver to be released. The snap detent 353 may then engage the snap engagement lip 355 to prevent the endoscope receiver 311 from rotating away from the locked position.

At least portions of the endoscope receiver 311, such as the locking snap 352, and at least portions of the cartridge base 331, such as the snap receiver 354, may formed from a semi-rigid, resilient material that permits deflection and reversion of the locking snap 352 and/or snap receiver 354. For example, portions of the endoscope receiver 311 and cartridge base 331 may be formed from a type of resilient plastic, such as acrylonitrile-butadiene-styrene (ABS). Further, the structure of locking snap 352 and/or cartridge base 331 may be designed to permit deflection and reversion of the locking snap 352 and/or cartridge base 331 when these two elements are caused to interfere with one another during rotation of the endoscope receiver 311. For example, the thickness, length, and/or other structural properties of the locking snap 352 and/or snap receiver 354 may be selected to allow limited deflection. The endoscope receiver 311 (and/or a portion or majority thereof) may also be made of a translucent material such that the proximal end 314 of the endoscope 306 may be seen within the cartridge 304 even when the cartridge 304 is closed or locked.

When the detachable cartridge 304 is in the locked configuration, elements of the electrical interfaces 323A-B are electrically connected, and the cartridge drives 325A-B are engaged with the drums of the steerable endoscope 306. As described above regarding FIGS. 1A-B and 2A-B, the cartridge drives 325A-B and/or endoscope drums may include force-transmitting/receiving, alignment, and/or other features that promote alignment and engagement between the cartridge drives 325A-B and endoscope drums 322A-B for control of the endoscope steerable tip.

Further, when the detachable cartridge 304 is in the locked configuration, the endoscope receiver 311 and/or cartridge base 331 may exert retention force, such as a clamping, squeezing, or other type of retention force, on the endoscope steerable tip 314, to prevent the steerable endoscope 306 from being inadvertently withdrawn, or partially or wholly detached from the detachable cartridge 302. Connection between electrical interfaces 323A-B and engagement of the cartridge drives 325A-B and endoscope drums may help prevent detachment/withdraw of the steerable endoscope 306 from the detachable cartridge 304, as described above.

A surface of the endoscope receiver 311 may include a lock indicator 321, which provides a visual indication that the detachable cartridge 304 is locked, and that the endoscope proximal end 314 is retained in the detachable cartridge 304. In some examples, the detachable cartridge 304 may include an unlock indicator that indicates the detachable cartridge is unlocked and that the steerable endoscope 306 may be removed from the endoscope receiver 311.

To remove the steerable endoscope 306 from the detachable cartridge 304, the locking snap 352 is depressed so that the snap detent 353 clears the snap engagement lip 355, allowing the endoscope receiver 311 to be rotated away from the cartridge base 331. Force may then be applied to the endoscope proximal end 314 to overcome retention force exerted between the endoscope receiver 311 and the endoscope proximal end 314 (such as forces applied by press-fit), thereby releasing the endoscope proximal end 314 from the endoscope receiver 311. In examples, the detachable cartridge 304 may include one or more spring elements that facilitate opening/unlocking of the detachable cartridge 304, such as by applying force that facilitates rotation of the endoscope receiver 311 from the closed/locked position to the open/unlocked position. For instance, one or more spring elements associated with the receiver hinge 356, cartridge base 331, and/or endoscope receiver 311 may apply rotational force to the endoscope receiver 311 that facilitates unlocking of the detachable cartridge 304 when the locking snap 352 is depressed.

FIG. 3C depicts the detachable cartridge 304 (with steerable endoscope 306 removed) in the locked configuration, connected to the VL 302. The VL 302 may include similar, or the same, elements, features, and/or functions as VL 102 and 202 described above. For example, the VL 302 may include a handle 308, rear surface 303, drive housing 320, and display 312, which may each include the same features and/or elements, and/or may perform the same functions as described for VL 102 and 202. As another example, the VL 302 and/detachable cartridge 304 may include one or more permanent magnets for aligning and retaining the detachable cartridge 304 to the VL 302.

In addition, the VL 302 includes an electrical interface that connects to electrical interface 323C on a cartridge interface surface 315 (depicted in FIG. 3D) of the cartridge base 331 when the detachable cartridge 304 is connected to the VL 302. VL 302 includes mechanical drives that engage mechanical receivers 324A-B and transmit steering forces generated within the VL 302 (such as by motors or other actuators). As described, elements of the mechanical drives of the VL 302 and cartridge mechanical receivers 324A-B may include force-transmitting/receiving, alignment, and/or other features promote alignment and engagement between the mechanical drives of the VL 302 and cartridge mechanical receivers 324A-B.

In examples, the VL 302, detachable cartridge 304, and steerable endoscope 306 may be connected and disconnected in any order, at any time during operation. For example, the steerable endoscope 306 may be removed from the detachable cartridge 304 in order to post-load a breathing tube, while the detachable cartridge 304 remains coupled to the VL 302. The steerable endoscope 306 may be reinserted into the detachable cartridge 304 following the post-load procedure. In another example, the detachable cartridge 304 may be decoupled from the VL 302 while the steerable endoscope 306 remains coupled to the detachable cartridge 304.

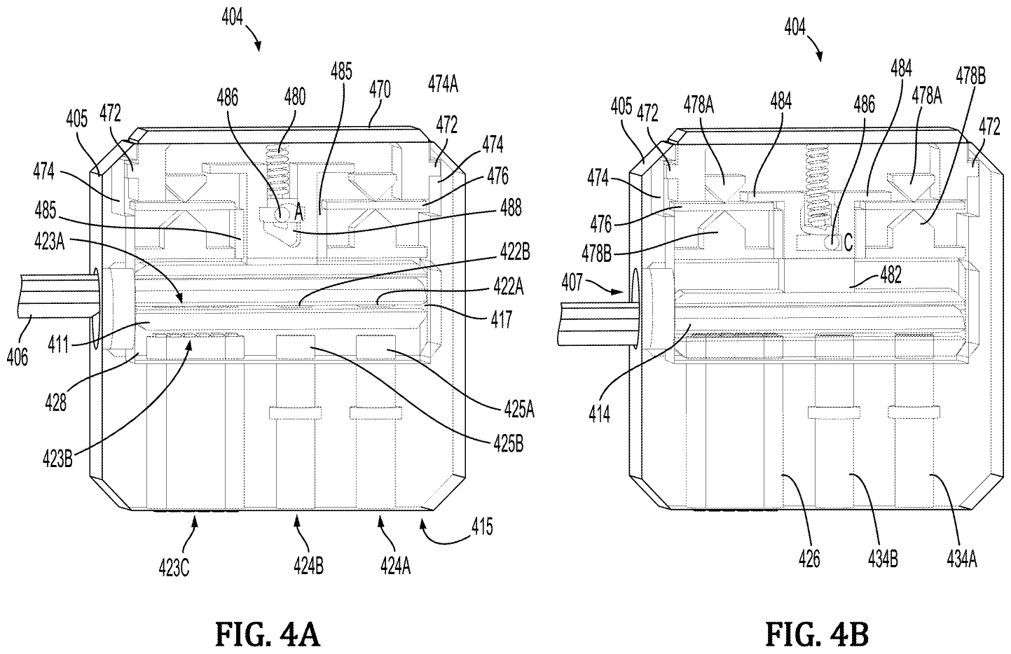

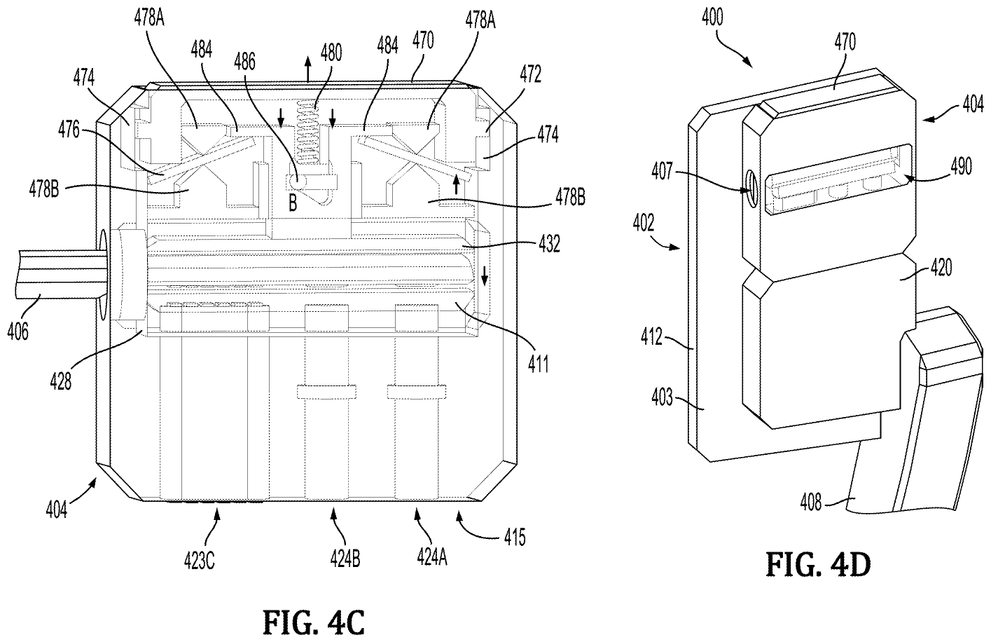

FIG. 4A-D depict another example of a detachable cartridge 404 that uses a push-button locking system for connecting a steerable endoscope 406 to the detachable cartridge 404. FIG. 4A depicts an example push-button detachable cartridge 404 in an open or unlocked configuration. FIG. 4B depicts an example push-button detachable cartridge 404 in a closed or locked configuration. FIG. 4C depicts an example push-button detachable cartridge 404 in a state of transition from the unlocked configuration to the locked configuration. FIG. 4D depicts an example VL system 400 with the example twist-lock detachable cartridge 404 connected.

The detachable cartridge 404 and steerable endoscope 406 may include elements and features similar to, or the same as, elements and features described above for detachable cartridges 104, 204, and 304, and steerable endoscopes 106, 206, and 306. For example, elements of the electrical interfaces 423A-C may be similar to, and/or function the same as, electrical interfaces 123A-C, 223A-C, and/or 323A-C. The detachable cartridge 404 include an electrical passthrough 426 that connects electrical interfaces 423B-C, and that may be similar to, or the same as, electrical passthrough 126.

Further, steering control elements such as mechanical receivers 424A-B and cartridge drives 425A-B may be similar to, and/or function the same as, the steering control elements 124A-B/224A-B and 125A-B/225A-B. The steerable endoscope 406 also includes drums 422A-B that may be similar to, or the same as, drums 122A-B and/or 222A-B, which may be mechanically linked to, and provide for control of, the endoscope steerable tip.

The detachable cartridge 404 includes an endoscope receiver or receptacle 411 positioned in an inner chamber 428 within an exterior housing 405. The endoscope receiver 411 includes a cavity that accepts the endoscope proximal end 414, which is inserted into the endoscope receiver 411 through endoscope port 407. The endoscope receiver 411 includes a receiver stop 417 that is abutted by the endoscope proximal end 414 when the endoscope proximal end 414 is fully inserted into the endoscope receiver 411. In some examples, the receiver stop 417 may be the terminus of the cavity of the endoscope receiver 411 that accepts the endoscope proximal end 114 (as depicted in FIG. 4A), while in other examples the receiver stop 417 may a defined mechanical stop or similar element. As described for the above locking mechanisms, the receiver stop 417 prevents further insertion of the endoscope proximal end 414, when the electrical interfaces 423A-B are aligned, and endoscope drums 422A-B are aligned with cartridge drives 425A-B.

The dimensions of the inner chamber 428 may be chosen to allow limited, bidirectional travel of the endoscope receiver 411 in directions parallel to the mechanical couplings 434A-B. In examples, the endoscope receiver 411 and/or inner chamber 428 may include features, such as guides, rails, grooves, and/or other features associated with restricting, directing, and/or facilitating bidirectional travel of the endoscope receiver 411.

Locking and unlocking of the cartridge 304 may be achieved by pressing of a push button 470 that causes movement of a plunger 482 and plunger platform 432 that ultimately moves the proximal end 414 of the endoscope 406. The endoscope receiver 411 is coupled to a plunger 482, which is restricted by plunger guides 485 to bidirectional travel that is parallel to the plunger guides 485, which in turn are parallel to the mechanical couplings 434A-B. Force is exerted on the plunger 482 by spring 480 in the direction of travel. The spring 480 is further connected to the push button 470, to which the spring 480 applies a counteracting force away from the plunger 482. The push button includes button tabs 472 that travel within tab guides 474 and restrict the push button 470 to bidirectional travel that is parallel to the tab guides 474, which in turn may be oriented parallel to the mechanical couplings 434A-B. In the locked configuration (depicted in FIG. 4A) and the unlocked configuration (depicted in FIG. 4B) the button tabs 472 abut an upper portion of the tab guides 474 (or a mechanical stop or similar element) to prevent further travel of the push button 470 away from the plunger 482. A lower portion of the tab guides 474 may prevent travel of the push button 470 towards the plunger 482.

The plunger 482 is also associated with a ball bearing 486 whose travel is restricted within a bearing race 488. In the locked configuration, the ball bearing 486 is positioned within the bearing race 488 at location A, depicted in FIG. 4A. Portions of the plunger 482 engage the ball bearing 486, which acts as a mechanical stop to maintain the plunger 482 and endoscope receiver 411 in the unlocked configuration. In some examples, the bearing race 488 may include a detent or similar feature that allows the ball bearing 486 to be retained at location A until released by further actuation of the plunger 482 via the push button 470.

To configure the detachable cartridge 404 in the locked configuration (as depicted in FIG. 1C), the push button 470 is depressed towards the cartridge interface surface 415, away from the starting/initial position of the push button 470 (depicted in FIGS. 4A-B). As the push button 470 advances, the push button 470 applies force to one side each of a pair of pivot arms 476, which rotate about pivot points created by upper and lower pivot mounts 478A-B. With further travel of the push button 470, a portion of the pivot arms 476 engages the plunger arms 484 and lifts the plunger 482 towards the push button 470. A portion of the plunger 482 engages the ball bearing 486, releasing the spring force from the ball bearing 486, and guiding travel of the ball bearing 486 in a counterclockwise direction around the bearing race 488. When the push button 470 is maximally depressed towards the steerable endoscope 406, the push button 470 may be released, allowing the spring 480 to force the push button 470 away from the plunger 482.

Further actuation of the push-button locking apparatus is depicted in FIG. 4C. Continued travel of the push button 470 away from the plunger 482 releases the force applied to the pivot arms 476, which subsequently releases force applied to the plunger arms 484, allowing the plunger 482 to advance towards the cartridge interface surface 415 (due to force applied by the spring 480). As the plunger 482 advances, the ball bearing 486 is caused to continue counterclockwise travel around the bearing race 488. The ball bearing 486 is depicted in FIG. 4C at an intermediate location B, as the detachable cartridge 404 is transitioning to the locked configuration. Travel of the plunger 482 towards the cartridge interface surface 415 causes the endoscope receiver 411 to advance towards the cartridge interface surface 415.

Returning to FIG. 4B, when the ball bearing 486 reaches a lower portion of the bearing race 488, further travel in the counterclockwise direction is prevented by the curvature of the bearing race 488. The ball bearing 486 comes to rest at location C, where the ball bearing 486 prevents further travel of the endoscope receiver 411. The force exerted on the plunger 482 by the spring 480 is transmitted to the ball bearing 486 by the plunger 482. This force helps maintain the ball bearing 486 at location C. In this configuration (the locked configuration), the electrical interfaces 423A-B are electrically connected, and the cartridge drives 425A-B are engaged with endoscope drums 422A-B, respectively. As described above for the preceding drawings, when the detachable cartridge 404 is in the locked configuration, elements of the detachable cartridge 404 may exert retention force (such as a clamping force, squeezing force, etc.) on the endoscope proximal end 414, to prevent the endoscope proximal end 414 from being unintentionally disconnected from the detachable cartridge 404.

In the locked configuration, the push button 470 is returned to the starting/initial position depicted in FIG. 4B. The detachable cartridge 404 may be transitioned from the locked configuration to the unlocked configuration by depressing the push button 470 again, whereby the pivot arms 476 again apply force to the plunger arms 484, causing the plunger 482 and endoscope receiver 411 to be lifted towards the push button 470. Travel of the plunger 482 towards the push button 470 causes the ball bearing 486 to travel counterclockwise along the bearing race 488, and come to rest at location A, depicted in FIG. 4A.

FIG. 4D depicts the example push-button detachable cartridge 404 (unlocked configuration) connected to a VL 402, which may include elements similar to, or the same as, elements described above for VL 102, 202, and 302. For example, the VL 402 includes a handle 408, display 412, and drive housing 420, which may each include elements and features, and/or perform functions, similar to, or the same as, corresponding elements, features, and/or functions as those described above. The VL 402 includes an electrical interface that electrically connects to cartridge-VL electrical interface 423C, and mechanical drives that engage cartridge mechanical receivers 424A-B. The electrical interface of the VL 402 may distribute electrical power to the detachable cartridge 404 and/or steerable endoscope 406 and provide a signal path for transmitting/receiving electrical signals from the detachable cartridge 404 and/or steerable endoscope 406. The VL 402 may include motors or other actuators for controlling the endoscope steerable tip, via mechanical elements of the detachable cartridge 404.

Further, the VL 402 and/or detachable cartridge 404 may include one or more magnetic elements (such as permanent magnets) that align and retain the detachable cartridge 404 to the drive housing 420 and/or rear surface 403. In examples, the VL 402 and/or detachable cartridge 404 may include other types of elements for alignment and retention of the detachable cartridge 404. As shown in FIG. 4D, in some examples, the cartridge 404 may include a translucent window 490 in the rear of the cartridge such that a user can visually see if the endoscope 406 is coupled to the cartridge 404 (e.g., the endoscope electrical interface 423A is connected to the cartridge-endoscope electrical interface 423B and/or the drums 422A-B are connected to the mechanical cartridge drives 425A-B.

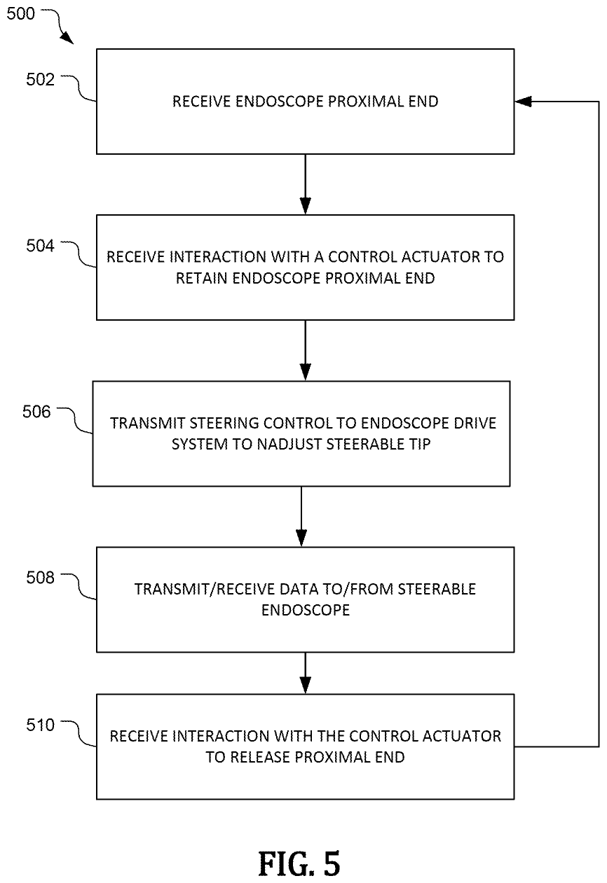

FIG. 5 depicts an example method 500 for retaining an endoscope in a detachable cartridge. At operation 502, an endoscope proximal end is received by the detachable cartridge. At operation 504, an interaction with a control actuator to lock the proximal end of the endoscope into the detachable cartridge is received. For instance, a manual interaction of a knob, slider, push button, or snap of the particular detachable cartridge may be received. The interaction and movement of the control actuator causes the proximal end of the endoscope to be locked (e.g., retained) in the cartridge.

At operation 506, steering control is transmitted to the drive system (e.g., drums) of the proximal end of the endoscope to adjust (e.g., steer) the adjustable tip of the endoscope. For instance, the mechanical force is transferred form the VL to drive system of the endoscope via the cartridge drives of the detachable cartridge. At operation 508, data is transferred between the VL and the endoscope via the cartridge. For instance, the cartridge may receive video or sensor data from the endoscope and pass the video or sensor data to the VL. At operation 510, another interaction with the control actuator (e.g., knob, slider, snap, button) to unlock the cartridge and release the proximal end of the endoscope. The method 500 may then repeat to reconnect the steerable endoscope to the cartridge.

Those skilled in the art will recognize that the methods and systems of the present disclosure may be implemented in many manners and as such are not to be limited by the foregoing aspects and examples. Any number of the features of the different aspects described herein may be combined into single or multiple aspects, and alternate aspects having fewer than or more than all of the features herein described are possible. Functionality may also be, in whole or in part, distributed among multiple components, in manners now known or to become known. Further, as used herein and in the claims, the phrase “at least one of element A, element B, or element C” is intended to convey any of: element A, element B, element C, elements A and B, elements A and C, elements B and C, and elements A, B, and C.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A cartridge, detachable from a video laryngoscope, that retains a proximal end of an endoscope, the cartridge comprising:
a housing;
an endoscope receptacle at least partially housed within the housing and shaped to receive the proximal end of the endoscope;
an electrical interface exposed inside the housing; and
an actuator having a locked and unlocked position, wherein adjustment of the actuator from the unlocked position to the locked position:
moves the proximal end of the endoscope into contact with the electrical interface; and
locks the proximal end of the endoscope in the cartridge.

2. The cartridge of claim 1, further comprising a drive, wherein adjustment of the actuator from the unlocked position to the locked position couples a drum of the endoscope with the drive.

3. The cartridge of claim 2, further comprising a mechanical receiver connected to the drive, wherein the mechanical receiver is exposed on a cartridge surface that contacts the video laryngoscope when the cartridge is attached to the video laryngoscope.

4. The cartridge of claim 1, wherein the actuator is a slide.

5. The cartridge of claim 4, further comprising:
a mount; and
a retention track, wherein as the slide travels along the mount, the retention track causes the endoscope receptacle to move.

6. The cartridge of claim 1, wherein the actuator is a knob.

7. The cartridge of claim 6, further comprising:
a rotary guide;
a retention shaft; and
a thread traveler.

8. The cartridge of claim 1, wherein the actuator is a push button.

9. The cartridge of claim 8, further comprising:
a spring coupled to the push button; and
a plunger coupled to the spring, wherein depression of the push button causes the plunger to move the proximal end of the endoscope.

10. A cartridge, detachable from a video laryngoscope, that retains a proximal end of an endoscope, the cartridge comprising:
a receptacle shaped to receive the proximal end of the endoscope; and
at least one of a knob, a slide, a push button, or a locking snap, wherein movement of the at least one of the knob, the slide, the push button, or the locking snap from an unlocked position to a locked position causes the proximal end of the endoscope to be locked in the receptacle.

11. The cartridge of claim 10, wherein the cartridge includes an electrical interface, and the movement of the at least one of the knob, the slide, the push button, or the locking snap causes an electrical interface of the endoscope to contact the electrical interface of the cartridge.

12. The cartridge of claim 11, wherein the cartridge includes a drive, and the movement of the at least one of the knob, the slide, the push button, or the locking snap causes a drum of the endoscope to contact the drive.

13. The cartridge of claim 12, further comprising a mechanical stop at an end of the receptacle, wherein when the proximal end of the endoscope contacts the mechanical stop:
the drive is aligned with the drum; and
the electrical interface of the cartridge is aligned with the electrical interface of the endoscope.

14. A system for retaining an endoscope in a cartridge, detachable from a video laryngoscope, the system comprising:
an endoscope comprising a steerable distal end and a drive system for steering the steering the steerable distal end; and
a cartridge, detachable from a video laryngoscope, comprising:
an endoscope receptacle shaped to receive the drive system of the endoscope;
a drive; and
an actuator having a locked and unlocked position, wherein adjustment of the actuator from the unlocked position to the locked position:
moves the drive system of the endoscope into contact with the drive; and
locks the drive system of the endoscope in the cartridge.

15. The system of claim 14, wherein the actuator is one of a knob, a slide, a push button, or a locking snap.
